(19) United States
(12) Patent Application Publication
(10) Pub. No.: US 2023/0167070 A1
(43) Pub. Date: Jun. 1, 2023

(54) MODULATORS OF TREX1

(71) Applicant: Constellation Pharmaceuticals, Inc., Boston, MA (US)

(72) Inventors: Julian R. Levell, Arlington, MA (US); Jonathan E. Wilson, Arlington, MA (US); Aaron Coffin, Everett, MA (US); Kennedy Taveras, Lynn, MA (US)

(21) Appl. No.: 17/922,388

(22) PCT Filed: Apr. 30, 2021
(86) PCT No.: PCT/US2021/030189

§ 371 Date: Oct. 31, 2022

Related U.S. Application Data
(60) Provisional application No. 63/018,808, filed on May 1, 2020.

(51) Int. Cl. C07D 239/14; A61P 35/00

(52) U.S. Cl. CPC C07D 239/14; A61P 35/00

(57) ABSTRACT

Provided are compounds of Formula (I): and pharmaceutically acceptable salts and compositions thereof, which are useful for treating a variety of conditions associated with TREX1.

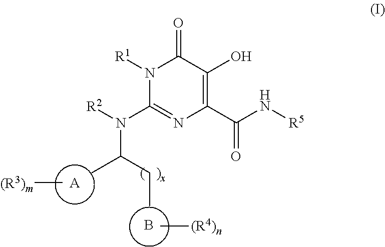

### RELATED APPLICATIONS

This application claims the benefit of priority to U.S. Provisional Application No. 63/018,808, filed May 1, 2020, the entire contents of which are incorporated herein by reference.

### BACKGROUND

A potential immune therapy is needed for cancers related to the innate immune system recognition of non-self, and to detect and protect against potential danger. Cancer cells differ antigenically from their normal counterparts and emit danger signals to alert the immune system similar to viral infection. These signals, which include damage-associated molecular patterns (DAMPs) and pathogen-associated molecular patterns (PAMPs), further activate the innate immune system resulting in the protection of the host from a variety of threats (Front. Cell Infect. Microbiol. 2012, 2, 168).

Ectopically expressed single stranded DNA (ssDNA) and double stranded DNA (dsDNA) are known PAMPs and/or DAMPs, which are being recognized by the cyclic GMP-AMP synthase (cGAS), a nucleic acid sensor (Nature 2011, 478, 515-518). Upon sensing of cytosolic DNA, cGAS catalyzes the generation of the cyclic dinucleotide 2′,3′-cGAMP, a potent second messenger and activator of the ER transmembrane adapter protein stimulator of interferon genes (STING) (Cell Rep. 2013, 3, 1355-1361). STING activation triggers phosphorylation of IRF3 via TBK1 which in turn leads to type I interferon production and activation of interferon stimulated genes (ISGs); a pre-requisite to the activation of innate immunity and initiation of adaptive immunity. Production of type I interferons thus constitutes a key bridge between the innate and adaptive immunity (Science 2013, 341, 903-906).

Excess type I IFN can be harmful to the host and induce autoimmunity, therefore, negative feedback mechanisms exist that keep type I IFN-mediated immune activation in check. Three prime repair exonuclease I (TREX1) is a 3′-5′ DNA exonuclease responsible for the removal of ectopically expressed ssDNA and dsDNA and is therefore a key repressor of the cGAS/STING pathway (PNAS 2015, 112, 5117-5122).

Type I interferons and downstream pro-inflammatory cytokine responses are critical to the development of immune responses and their effectiveness. Type I interferons enhance both the ability of dendritic cells and macrophages to take up, process, present, and cross-present antigens to T cells, and their potency to stimulate T cells by eliciting the upregulation of the co-stimulatory molecules such as CD40, CD80 and CD86 (J. Exp. Med. 2011, 208, 2005-2016). Type I interferons also bind their own receptors and activate interferon responsive genes that contribute to activation of cells involved in adaptive immunity (EMBO Rep. 2015, 16, 202-212).

From a therapeutic perspective, type I interferons and compounds that can induce type I interferon production have potential for use in the treatment of human cancers (Nat. Rev Immunol. 2015, 15, 405-414). Interferons can inhibit human tumor cell proliferation directly. In addition, type I interferons can enhance anti-tumor immunity by triggering the activation of cells from both the innate and adaptive immune system. Importantly, the anti-tumor activity of PD-1 blockade requires pre-existing intratumoral T cells. By turning cold tumors into hot and thereby eliciting a spontaneous anti-tumor immunity, type I IFN-inducing therapies have the potential to expand the pool of patients responding to anti-PD-1 therapy as well as enhance the effectiveness of anti-PD1 therapy.

Human and mouse genetic studies suggest that TREX1 inhibition might be amenable to a systemic delivery route and therefore TREX1 inhibitory compounds could play an important role in the anti-tumor therapy landscape. TREX1 is a key determinant for the limited immunogenicity of cancer cells responding to radiation treatment [Trends in Cell Biol., 2017, 27 (8), 543-4; Nature Commun., 2017, 8, 15618]. TREX1 is induced by genotoxic stress and involved in protection of glioma and melanoma cells to anticancer drugs [Biochim. Biophys. Acta, 2013, 1833, 1832-43]. STACT-TREX1 therapy shows robust anti-tumor efficacy in multiple murine cancer models [Glickman et al, Poster P235, 33^{rd} Annual Meeting of Society for Immunotherapy of Cancer, Washington D.C., Nov. 7-11, 2018]. (TREX1) expression correlates with cervical cancer cells growth in vitro and disease progression in vivo [Scientific Reports 1019, 9, 351]. Beyond oncology there is also support for agonists of the IFN pathway to be useful in antiviral therapy, for example STING agonists induce an innate antiviral immune response against Hepatitis B Virus via stimulation of the IFN pathway and upregulation of ISG's [Antimicrob. Agents Chemother. 2015, 59:1273-1281] and TREX1 inhibits the innate immune response to HIV type 1 [Nature Immunology, 2010, 11(11), 1005].

### SUMMARY

Provided herein are compounds having the Formula I:

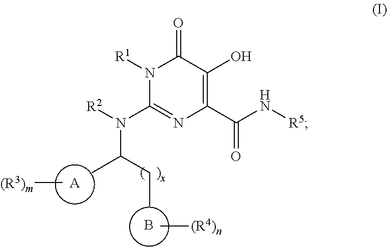

and pharmaceutically acceptable salts and compositions thereof, wherein R¹, R², R³, R⁴, R⁵, x, m, and n are as described herein. The disclosed compounds and compositions modulate TREX1, and are useful in a variety of therapeutic applications such as, for example, in treating cancer.

### DETAILED DESCRIPTION

### 1. General Description of Compounds

In a first embodiment, provided herein is a compound of Formula I:

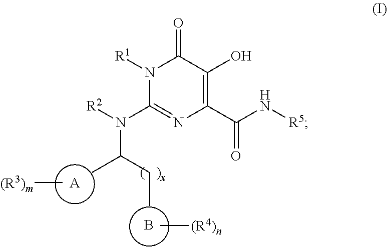

or a pharmaceutically acceptable salt thereof, wherein:

R¹ is hydrogen, (C₁-C₄)alkyl, halo(C₁-C₄)alkyl, or 3- to 4-membered cycloalkyl;

R² is hydrogen or (C₁-C₄)alkyl optionally substituted with phenyl, wherein said phenyl is optionally substituted with 1 to 3 groups selected from halo, (C₁-C₄)alkyl, and halo(C₁-C₄)alkyl;

Ring A and Ring B are each independently is aryl, heteroaryl, heterocyclyl, or cycloalkyl;

R³, R⁴, and R⁶ are each independently (C₁-C₆)alkyl, halo(C₁-C₆)alkyl, (C₁-C₆)alkylOR^{b}, (C₂-C₆)alkenyl, halo(C₁-C₆)alkoxy, halo, phenyl, —CN, —NR^{a}C(O)OR^{b}, —NR^{a}C(S)OR^{b}, —C(O)R^{b}, —NR^{a}C(O)NR^{b}R^{g}, —NR^{a}C(S)NR^{b}R^{g}, —NR^{a}S(O)₂NR^{b}R^{g}, —C(S)R^{b}, —S(O)₂R^{c}, —S(O)R^{c}, —C(O)OR^{d}, —C(S)OR^{d}, —C(O)NR^{e}R^{f}, —C(S)NR^{a}R^{e}, —NR^{a}C(O)R^{d}, —NR^{a}C(S)R^{d}, —OR^{e}, —SR^{e}, —O(C₁-C₄)alkylOR^{e}, —NR^{e}R^{f}, 4- to 6-membered heteroaryl, or 4- to 7-membered heterocyclyl, wherein
i) said phenyl for R³ and R⁴ are each independently optionally substituted with 1 or 2 groups selected from R^{g};
ii) said (C₁-C₆)alkyl for R³ and R⁴ are each independently optionally substituted with 1 or 2 groups selected from OR^{h}, —NR^{j}R^{k}, phenyl, and 5- to 6-membered heteroaryl; and
ii) said 4- to 7-membered heterocyclyl and 4- to 6-membered heteroaryl for R³ and R⁴ are each independently optionally substituted with 1 or 2 groups selected from R^{m}; and
iv) said phenyl and 5- to 6-membered heteroaryl of the optional substituents listed for (C₁-C₆)alkyl in R³ and R⁴ are each independently optionally substituted with 1 or 2 groups selected from R^{g};

x is 0, 1, or 2;

m and n are each independently an integer from 0 to 3;

R⁵ is (C₁-C₆)alkyl, halo(C₁-C₆)alkyl, (C₂-C₆)alkenyl, —(C₁-C₆)alkylOR^{a}, —(C₁-C₆)alkylNR^{a}R^{b}, —(C₁-C₆)alkyl(C₃-C₇)cycloalkyl, —(C₁-C₆)alkyl(4- to 7-membered heterocyclyl), —(C₁-C₆)alkyl(5- to 7-membered heteroaryl), phenyl, 5- to 7-membered heteroaryl, (C₃-C₇)cycloalkyl, (C₃-C₇)cycloalkenyl, or 4- to 7-membered heterocyclyl, wherein each occurrence of said phenyl, 5- to 7-membered heteroaryl, (C₃-C₇)cycloalkyl, (C₃-C₇)cycloalkenyl, or 4- to 7-membered heterocyclyl are optionally and independently substituted with 1 to 3 groups selected from R⁶, provided R⁵ is not an optionally substituted isoxazolyl;

R^{a}, R^{b}, R^{c}, R^{d}, R^{e} and R^{f} are each independently hydrogen, halo, (C₁-C₆)alkyl, halo(C₁-C₆)alkyl, (C₁-C₆)alkoxy, halo(C₁-C₆)alkoxy, phenyl, 3- to 4-membered cycloalkyl, 4- to 6-membered heteroaryl, or 4- to 7-membered heterocyclyl, wherein
i) said (C₁-C₆)alkyl for R^{a}, R^{b}, R^{c}, R^{d}, R^{e} and R^{f} is optionally substituted with 1 or 2 groups selected from phenyl, —OR^{h}, and —NR^{j}R^{k};
ii) said phenyl, 4- to 6-membered heteroaryl, and 4- to 7-membered heterocyclyl for R^{a}, R^{b}, R^{c}, R^{d}, R^{e}, and R^{f} are each optionally and independently substituted with 1 or 2 groups selected from R^{g}; and
iii) said 4- to 7-membered heterocyclyl for R^{a}, R^{b}, R^{c}, R^{d}, R^{e}, and R^{f} is further optionally substituted with ═O; and R^{g}, R^{h}, R^{j}, R^{k}, and R^{m} are each independently hydrogen, halo, (C₁-C₆)alkyl, halo(C₁-C₆)alkyl,

(C₁-C₆)alkoxy, halo(C₁-C₆)alkoxy, phenyl, —(C₁-C₆)alkylphenyl, 3- to 4-membered cycloalkyl, 4- to 6-membered heteroaryl, or 4- to 7-membered heterocyclyl, and wherein said 4- to 7-membered heterocyclyl for R^{g}, R^{h}, R^{j} and R^{k} is further optionally substituted with ═O.

### 2. Definitions

When used in connection to describe a chemical group that may have multiple points of attachment, a hyphen (-) designates the point of attachment of that group to the variable to which it is defined. For example, —NHC(O)OR^{a} and —NHC(S)OR^{a} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical.

The term “alkenyl” when used alone or as part of a larger moiety, means straight-chain or branched monovalent hydrocarbon radical having 1 or 2 double bonds.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term “aryl” refers to an aromatic carbocyclic ring system having, unless otherwise specified, a total of 6 to 10 ring members. In certain embodiments, “aryl” refers to an aromatic ring system which includes, but is not limited to, phenyl and naphthyl. It will be understood that when specified, optional substituents on an aryl group may be present on any substitutable position and, include, e.g., the position at which the aryl is attached.

The term “heteroaryl” used alone or as part of a larger moiety refers to a 5- to 12-membered (e.g., a 4- to 6-membered) aromatic radical containing 1-4 heteroatoms selected from N, O, and S. A heteroaryl group may be mono- or bi-cyclic as size permits. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “cycloalkyl” refers to a saturated cyclic hydrocarbon having from, unless otherwise specified, 3 to 10 carbon ring atoms. Monocyclic cycloalkyl groups include, without limitation, cyclopropyl, cyclobutyl, cyclopentyl, cyclohexyl, cycloheptyl, and cyclooctyl. It will be understood that when specified, optional substituents on a cycloalkyl or may be present on any substitutable position and, include, e.g., the position at which the cycloalkyl is attached.

The term “cycloalkenyl”, refers to a partially saturated cyclic hydrocarbon having from, unless otherwise specified, from 3 to 10 carbon atoms. Cycloalkenyl groups include, without limitation, cyclopropenyl, cyclobutenyl, cyclopentenyl, cyclohexenyl and cycloheptenyl. It will be understood that when specified, optional substituents on a cycloalkenyl group may be present on any substitutable position and, include, e.g., the position at which the cycloalkenyl group is attached.

The disclosed compounds exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. “R” and “S” represent the configuration of substituents around one or more chiral carbon atoms.

“Racemate” or “racemic mixture” means a compound of equimolar quantities of two enantiomers, wherein such mixtures exhibit no optical activity, i.e., they do not rotate the plane of polarized light.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the compound has one chiral center, it is to be understood that the name or structure encompasses one enantiomer of compound free from the corresponding optical isomer, a racemic mixture of the compound, or mixtures enriched in one enantiomer relative to its corresponding optical isomer.

The term “TREX1” refers to three prime repair exonuclease 1 or DNA repair exonuclease 1, which is an enzyme that in humans is encoded by the TREX1 gene. Mazur D J, Perrino F W (August 1999). “Identification and expression of the TREX1 and TREX2 cDNA sequences encoding mammalian 3′-->5′ exonucleases”. J Biol Chem. 274 (28): 19655-60. doi:10.1074/jbc.274.28.19655. PMID 10391904; Hoss M, Robins P, Naven T J, Pappin D J, Sgouros J, Lindahl T (August 1999). “A human DNA editing enzyme homologous to the Escherichia coli DnaQ/MutD protein”. EMBO J. 18 (13): 3868-75. doi:10.1093/emboj/18.13.3868. PMC 1171463. PMID 10393201. This gene encodes the major 3′->5′ DNA exonuclease in human cells. The protein is a non-processive exonuclease that may serve a proofreading function for a human DNA polymerase. It is also a component of the SET complex, and acts to rapidly degrade 3′ ends of nicked DNA during granzyme A-mediated cell death. Cells lacking functional TREX1 show chronic DNA damage checkpoint activation and extra-nuclear accumulation of an endogenous single-strand DNA substrate. It appears that TREX1 protein normally acts on a single-stranded DNA polynucleotide species generated from processing aberrant replication intermediates. This action of TREX1 attenuates DNA damage checkpoint signaling and prevents pathological immune activation. TREX1 metabolizes reverse-transcribed single-stranded DNA of endogenous retroelements as a function of cell-intrinsic antiviral surveillance, resulting in a potent type I IFN response. TREX1 helps HIV-1 to evade cytosolic sensing by degrading viral cDNA in the cytoplasm.

The term “TREX2” refers to Three prime repair exonuclease 2 is an enzyme that in humans is encoded by the TREX2 gene. This gene encodes a nuclear protein with 3′ to 5′ exonuclease activity. The encoded protein participates in double-stranded DNA break repair, and may interact with DNA polymerase delta. Enzymes with this activity are involved in DNA replication, repair, and recombination. TREX2 is a 3′-exonuclease which is predominantly expressed in keratinocytes and contributes to the epidermal response to UVB-induced DNA damage. TREX2 biochemical and structural properties are similar to TREX1, although they are not identical. The two proteins share a dimeric structure and can process ssDNA and dsDNA substrates in vitro with almost identical k_{cat} values. However, several features related to enzyme kinetics, structural domains, and subcellular distribution distinguish TREX2 from TREX1. TREX2 present a 10-fold lower affinity for DNA substrates in vitro compared with TREX1. In contrast with TREX1, TREX2 lacks a COOH-terminal domain that can mediate protein-protein interactions. TREX2 is localized in both the cytoplasm and nucleus, whereas TREX1 is found in the endoplasmic reticulum, and is mobilized to the nucleus during granzyme A-mediated cell death or after DNA damage.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, the compound of Formula I is of the Formula II:

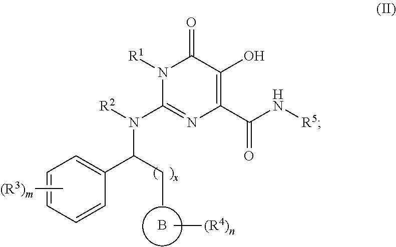

or a pharmaceutically acceptable salt thereof, wherein the variables are as described above for Formula I.

In a third embodiment, the compound of Formula I is of the Formula III:

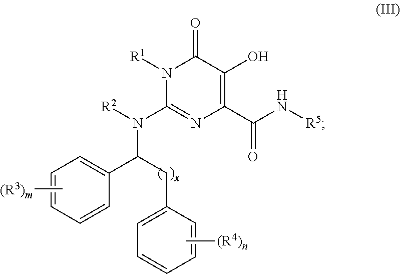

or a pharmaceutically acceptable salt thereof, wherein the variables are as described above for Formula I.

In a fourth embodiment, the compound of Formula I is of the Formula IV:

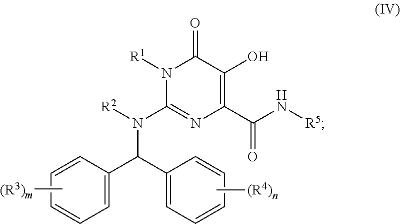

or a pharmaceutically acceptable salt thereof, wherein the variables are as described above for Formula I.

In a fifth embodiment, R² in the compounds of Formulae I, II, III, or IV is (C₁-C₄)alkyl, wherein the variables are as described above for Formula I.

In a sixth embodiment, the compound of Formula I is of the Formula V:

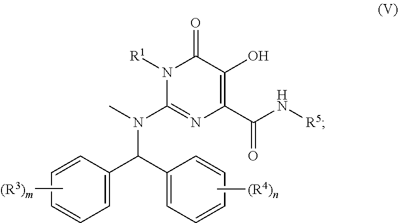

or a pharmaceutically acceptable salt thereof, wherein the variables are as described above for Formula I.

In a seventh embodiment, R¹ in the compounds of Formulae I, II, III, IV, or V is (C₁-C₄)alkyl, wherein the variables are as described above for Formula I or the fifth embodiment.

In an eighth embodiment, the compound of Formula I is of the Formula VI:

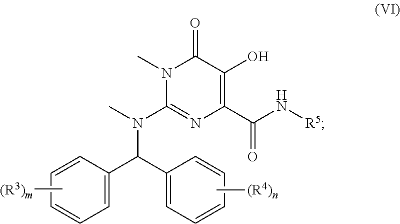

or a pharmaceutically acceptable salt thereof, wherein the variables are as described above for Formula I.

In a ninth embodiment, R³ and R⁴ in the compounds of Formulae I, II, III, IV, V, or VI are independently selected from (C₁-C₄)alkyl, halo(C₁-C₄)alkyl, (C₁-C₄)alkoxy, halo(C₁-C₄)alkoxy, halo and CN, wherein the variables are as described above for Formula I or the fifth or seventh embodiment.

In a tenth embodiment, R³ in the compounds of Formulae I, II, III, IV, V, or VI is halo, wherein the variables are as described above for Formula I or the fifth, seventh, or ninth embodiment. Alternatively, as part of a tenth embodiment, R³ in the compounds of Formulae I, II, III, IV, V, or VI is chloro, wherein the variables are as described above for Formula I or the fifth, seventh, or ninth embodiment.

In an eleventh embodiment, m in the compounds of Formulae I, II, III, IV, V, or VI is 0 or 1, wherein the variables are as described above for Formula I or the fifth, seventh, ninth, or tenth embodiment.

In a twelfth embodiment, n in the compounds of Formulae I, II, III, IV, V, or VI is 0, wherein the variables are as described above for Formula I or the fifth, seventh, ninth, tenth, or eleventh embodiment.

In a thirteenth embodiment, R⁵ in the compounds of Formulae I, II, III, IV, V, or VI is (C₁-C₆)alkyl, halo(C₁-C₆)alkyl, (C₂-C₆)alkenyl, —(C₁-C₅)alkylOR^{a}, —(C₁-C₆)alkyl(C₃-C₇)cycloalkyl, phenyl, 5- to 7-membered heteroaryl, (C₃-C₇)cycloalkyl, (C₃-C₇)cycloalkenyl, 4- to 7-membered heterocyclyl wherein each occurrence of said phenyl, 5- to 7-membered heteroaryl, (C₃-C₇)cycloalkyl, (C₃-C₇)cycloalkenyl, or 4- to 7-membered heterocyclyl are optionally and independently substituted with 1 to 3 groups selected from R⁶, wherein the variables are as described above for Formula I or the fifth, seventh, ninth, tenth, eleventh, or twelfth embodiment. Alternatively, as part of a thirteenth embodiment, R⁵ in the compounds of Formulae I, II, III, IV, V, or VI is (C₁-C₄)alkyl, halo(C₁-C₄)alkyl, (C₃-C₅)alkenyl, —(C₁-C₄)alkylO(C₁-C₄)alkyl, —(C₁-C₄)alkyl(C₄-C₆)cycloalkyl, phenyl, 5- to 6-membered heteroaryl, (C₃-C₆)cycloalkyl, (C₄-C₆)cycloalkenyl, or 4- to 6-membered heterocyclyl wherein each occurrence of said phenyl, 5- to 6-membered heteroaryl, (C₃-C₆)cycloalkyl, (C₄-C₆)cycloalkenyl, or 4- to 6-membered heterocyclyl are optionally and independently substituted with 1 to 3 groups selected from R⁶, wherein the variables are as described above for Formula I or the fifth, seventh, ninth, tenth, eleventh, or twelfth embodiment. In another alternative, as part of a thirteenth embodiment, R⁵ in the compounds of Formulae I, II, III, IV, V, or VI is (C₁-C₄)alkyl, halo(C₁-C₄)alkyl, (C₃-C₅)alkenyl, —(C₁-C₄)alkylO(C₁-C₄)alkyl, —(C₁-C₄)alkylcyclopropyl, cyclobutyl, cyclopentenyl, cyclobutyl, phenyl, pyridyl, pyrimidyl, oxazolyl, thiazolyl, isothiazolyl, oxadiazolyl, pyrrolyl, pyrazole or tetrazolyl, wherein each of said cyclopropyl, cyclobutyl, cyclopentenyl, cyclobutyl, phenyl, pyridyl, pyrimidyl, oxazolyl, thiazolyl, isothiazolyl, oxadiazolyl, pyrrolyl, pyrazole and tetrazolyl are optionally and independently substituted with 1 to 3 groups selected from R⁶, wherein the variables are as described above for Formula I or the fifth, seventh, ninth, tenth, eleventh, or twelfth embodiment. In another alternative, as part of a thirteenth embodiment, R⁵ in the compounds of Formulae I, II, III, IV, V, or VI is phenyl, pyridyl, pyrimidyl, oxazolyl, thiazolyl, isothiazolyl, oxadiazolyl, pyrrolyl, pyrazole or tetrazolyl, each of which is optionally and independently substituted with 1 to 3 groups selected from R⁶, wherein the variables are as described above for Formula I or the fifth, seventh, ninth, tenth, eleventh, or twelfth embodiment.

In a fourteenth embodiment, R⁶ in the compounds of Formulae I, II, III, IV, V, or VI is (C₁-C₆)alkyl, halo(C₁-C₆)alkyl, (C₁-C₆)alkylOR^{b}, (C₂-C₆)alkenyl, O(C₁-C₄)alkylOR^{e}, halo(C₁-C₆)alkoxy, halo, —NR^{a}C(O)OR^{b}, —NR^{a}C(S)OR^{b}, —C(O)R^{b}, —NR^{a}C(O)NR^{b}R^{g}, —NR^{a}C(S)NR^{b}R^{g}, —NR^{a}S(O)₂NR^{b}R^{g}, —C(S)R^{b}, S(O)₂R^{b}, S(O)Rb^{c}, —C(O)OR^{b}, —C(S)OR^{b}, —C(O)NR^{e}R^{f}, —C(S)NR^{a}R^{e}, —NR^{a}C(O)R^{d}, —NR^{a}C(S)R^{b}, —OR^{e}, —SR^{e}, or —NR^{e}R^{f}, wherein the variables are as described above for Formula I or the fifth, seventh, ninth, tenth, eleventh, twelfth, or thirteenth embodiment.

In a fifteenth embodiment, R^{a}, R^{b}, R^{c}, R^{e}, R^{f}, and R^{g} in the compounds of Formulae I, II, III, IV, V, or VI are each independently hydrogen, (C₁-C₆)alkyl or halo(C₁-C₆)alkyl, wherein the variables are as described above for Formula I or the fifth, seventh, ninth, tenth, eleventh, twelfth, thirteenth, or fourteenth embodiment.

In a sixteenth embodiment, R⁶ in the compounds of Formulae I, II, III, IV, V, or VI is (C₁-C₄alkyl or halo(C₁-C₄)alkyl, wherein the variables are as described above for Formula I or the fifth, seventh, ninth, tenth, eleventh, twelfth, thirteenth, fourteenth, or fifteenth embodiment.

In one aspect of the compounds described herein, included any one of the disclosed embodiment, R⁵ is not an optionally substituted isoxazol-4-yl.

Compounds having the Formula I are further disclosed in the Exemplification and are included in the present disclosure. Pharmaceutically acceptable salts thereof as well as the neutral forms are included.

### 4. Uses, Formulation and Administration

Compounds and compositions described herein are generally useful for modulating the activity of TREX1. In some aspects, the compounds and pharmaceutical compositions described herein inhibit the activity TREX1.

In some aspects, compounds and pharmaceutical compositions described herein are useful in treating a disorder associated with TREX1 function. Thus, provided herein are methods of treating a disorder associated with TREX1 function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with TREX1 function. Also provided is a compound described herein, or a pharmaceutically acceptable salt thereof, or a pharmaceutical composition comprising a disclosed compound or pharmaceutically acceptable salt thereof, for use in treating a disorder associated with TREX1.

In some aspects, the compounds and pharmaceutical compositions described herein are useful in treating cancer.

In some aspects, the cancer treated by the compounds and pharmaceutical compositions described herein is selected from colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, and breast cancer.

In some aspects, the cancer treated by the compounds and pharmaceutical compositions described herein is selected from lung cancer, breast cancer, pancreatic cancer, colorectal cancer, and melanoma.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### Exemplification

### Chemical Synthesis

The representative examples that follow are intended to help illustrate the present disclosure, and are not intended to, nor should they be construed to, limit the scope of the invention.

General starting materials used were obtained from commercial sources or prepared in other examples, unless otherwise noted.

The following abbreviations have the indicated meanings:

Ac=acetyl; ACN=acetonitrile; AcO acetate; BOC=t-butyloxycarbonyl; CBZ=carbobenzoxy; CDI=carbonyldiimidazole; DBU=1,8-Diazabicycloundec-7-ene; DCC=1,3-dicyclohexylcarbodiimide; DCE=1,2-dichloroethane; DI=de-ionized; DIAD=Diisopropyl azodicarboxylate; DIBAL=diisobutyl aluminum hydride; DIPA=diisopropylamine; DIPEA or DIEA=N,N-diisoproylethylamine, also known as Hunig's base; DMA=dimethylacetamide; DMAP=4-(dimethylamino)pyridine; DMF=dimethylformamide; DMP=Dess-Martin periodinane; DPPA=Diphenylphosphoryl azide; DPPP=1,3-bis(diphenylphosphino)propane; Dtbbpy=4,4′-di-/e/7-butyl-2,2′-dipyridyl; EDC or EDCI 1-(3-dimethylaminopropyl)-3-ethylcarbodiimide hydrochloride; EDTA=ethylenediaminetetraacetic acid, tetrasodium salt; EtOAc=ethyl acetate; FAB=fast atom bombardment; FMOC=9-fluorenylmethoxycarbonyl; HMPA=hexamethylphosphoramide; HATU=(9-(7-Azabenzotriazol-1-yl)-N, N, N, N-tetramethyluroniumhexafluorophosphate; HOAt=1-Hydroxy-7-azabenzotriazole or 3H-[1,2,3]triazolo[4,5-b]pyridin-3-ol; HOBt=1-hydroxybenzotriazole; HRMS=high resolution mass spectrometry; KHMDS=potassium hexamethyldisilazane; LC-MS=Liquid chromatography-mass spectrometry; LDA=lithium diisopropylamide; LiHMDS=lithium hexamethyldisilazane; MCPBA=meta-chloroperbenzoic acid; MMPP=magnesium monoperoxyphthlate hexahydrate; Ms=methanesulfonyl=mesyl; MsO=methanefulfonate=mesylate; MTBE=Methyl t-butyl ether; NBS=N-bromosuccinimide; NMM=4-methylmorpholine; NMP=N-methylpyrrolidinone; NMR=Nuclear magnetic resonance; PCC=pyridinium chlorochromate; PDC=pyridinium dichromate; Ph=phenyl; PPTS=pyridinium p-toluene sulfonate; pTSA=p-toluene sulfonic acid; r.t./RT=room temperature; rac.=racemic; T3P=2,4,6-Tripropyl-1,3,5,2,4,6-trioxatriphosphinane 2,4,6-trioxide; TEA=triethylamine; TFA=trifluoroacetic acid; TfO=trifluoromethanesulfonate=triflate; THF=tetrahydrofuran; TLC=thin layer chromatography; TMSCl=trimethylsilyl chloride.

The progress of reactions was often monitored by TLC or LC-MS. The LC-MS was recorded using one of the following methods.

LCMS METHOD-1:

| Mobile | (A) | 2 mM Ammonium acetate + | | |
|---|---|---|---|---|
| Phase | | 0.1% Formic Acid in Water | | |
| | (B) | 0.1% Formic Acid in Acetonitrile | | |
| Column | : | BEH C18 (50 * 2.1 mm) 1.7 um | | |
| Column | : | 0.55 ml/min | | |
| Flow | | | | |
| Gradient | : | Time (min) | % A | % B |
| | | 0.01 | 98 | 2 |
| | | 0.30 | 98 | 2 |
| | | 0.60 | 50 | 50 |
| | | 1.10 | 25 | 75 |
| | | 2.00 | 0 | 100 |
| | | 2.70 | 0 | 100 |
| | | 2.71 | 98 | 2 |
| | | 3.00 | 98 | 2 |

LCMS Method-2:

| Mobile | (A) | 5 mM Ammonium Acetate + | | |
|---|---|---|---|---|
| Phase | | 0.1% Formic Acid in Water | | |
| | (B) | 0.1% Formic Acid in Acetonitrile | | |
| Column | : | BEH C18 (50*2.1mm), 1.7 um or Equivalent | | |
| Column | : | 0.45 ml/min | | |
| Flow | | | | |
| Gradient | : | Time (min) | % A | % B |
| | | 0.01 | 98 | 2 |
| | | 0.50 | 98 | 2 |
| | | 5.00 | 10 | 90 |
| | | 6.00 | 5 | 95 |
| | | 7.00 | 5 | 95 |
| | | 7.01 | 98 | 2 |
| | | 8.00 | 98 | 2 |

LCMS Method-3:

| Mobile Phase | (A) | 5 mM Ammonium bicarbonate in water | | |
|---|---|---|---|---|
| | (B) | Acetonitrile | | |
| Column | : | X-Bridge C18 (50 * 4.6 mm), 3.5 um | | |
| Column Flow | : | 1.0 ml/min | | |
| Gradient | : | Time (min) | % A | % B |
| | | 0.01 | 95 | 5 |
| | | 5.00 | 10 | 90 |
| | | 5.80 | 5 | 95 |
| | | 7.20 | 5 | 95 |
| | | 7.21 | 95 | 5 |
| | | 10.00 | 95 | 5 |

LCMS Method-4:

| Mobile Phase | (A) | 10 mM Ammonium Acetate in WATER | | |
|---|---|---|---|---|
| | (B) | 100% Acetonitrile | | |
| Column | : | X-Bridge C18 (150 * 4.6 mm), | | |
| | | 5 um or Equivalent | | |
| Column Flow | : | 1.0 ml/min | | |
| Gradient | : | Time (min) | % A | % B |
| | | 0.01 | 90 | 10 |
| | | 5.00 | 10 | 90 |
| | | 7.00 | 0 | 100 |
| | | 11.00 | 0 | 100 |
| | | 11.01 | 90 | 10 |
| | | 12.00 | 90 | 10 |

LCMS Method-5:

| Mobile Phase | (A) | 10 mM Ammonium Acetate in Water | | |
|---|---|---|---|---|
| | (B) | 100% Acetonitrile | | |
| Column | : | X-Bridge C18 (150 * 4.6 mm), | | |
| | | 5 um or Equivalent | | |
| Column Flow | : | 1.0 ml/min | | |
| Gradient | : | Time (min) | % A | % B |
| | | 0.01 | 100 | 0 |
| | | 7.00 | 50 | 50 |
| | | 9.00 | 0 | 100 |
| | | 11.00 | 0 | 100 |
| | | 11.01 | 100 | 0 |
| | | 12.00 | 100 | 0 |

LCMS Method-5:

| Mobile Phase | (A) | 0.1% Formic Acid in Water | | |
|---|---|---|---|---|
| | (B) | 0.1% Formic Acid in Acetonitrile | | |
| Column | : | Zorbax SB-C8 (4.5 × 75 mm), 3.5 μm | | |
| Column Flow | : | 1.5 ml/min | | |
| Gradient | : | Time (min) | % A | % B |
| | | 0.00 | 95 | 5 |
| | | 3.60 | 5 | 95 |
| | | 4.00 | 5 | 95 |
| | | 4.50 | 95 | 5 |

NMR was recorded at room temperature unless noted otherwise on Varian Inova 400 or 500 MHz spectrometers with the solvent peak used as the reference or on Bruker 300 or 400 MHz spectrometers with the TMS peak used as internal reference.

The compounds described herein may be prepared using the following methods and schemes. Unless specified otherwise, all starting materials used are commercially available.

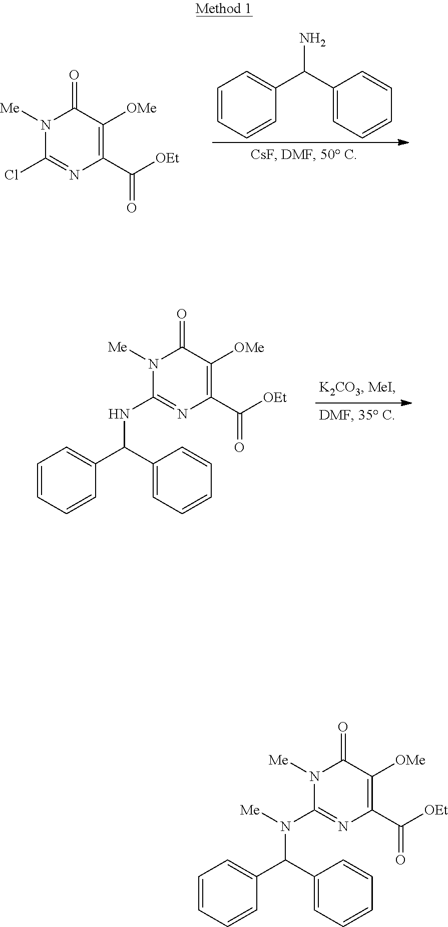 [SMILES: CCOC(=O)c1nc(Cl)n(C)c(=O)c1OC ; NC(c1ccccc1)c1ccccc1 ; CCOC(=O)c1nc(NC(c2ccccc2)c2ccccc2)n(C)c(=O)c1OC ; CCOC(=O)c1nc(N(C)C(c2ccccc2)c2ccccc2)n(C)c(=O)c1OC]

Method 1 is a 2-step protocol for the synthesis of ethyl 2-(benzhydryl(methyl)amino)-5-methoxy-1-methyl-6-oxo-1,6-dihydropyrimidine-4-carboxylate starting from ethyl 2-chloro-5-methoxy-1-methyl-6-oxo-1,6-dihydropyrimidine-4-carboxylate which is prepared following literature procedure. (Synthesis 2006, 8, 1343-1350)

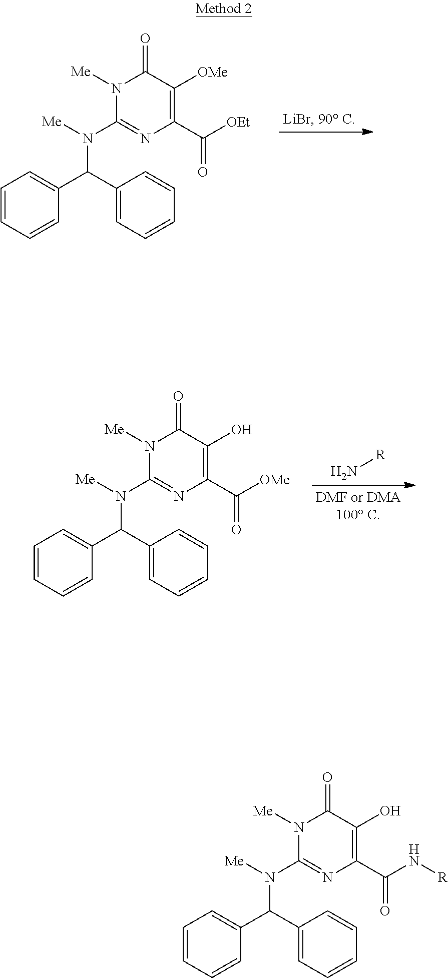

Method 2 is a 2-step protocol for the preparation of hydroxy pyrimidine analogs starting from ethyl 2-(benzhydryl(methyl)amino)-5-methoxy-1-methyl-6-oxo-1,6-dihydropyrimidine-4-carboxylate as prepared in method 1. This method applies to alkyl amines.

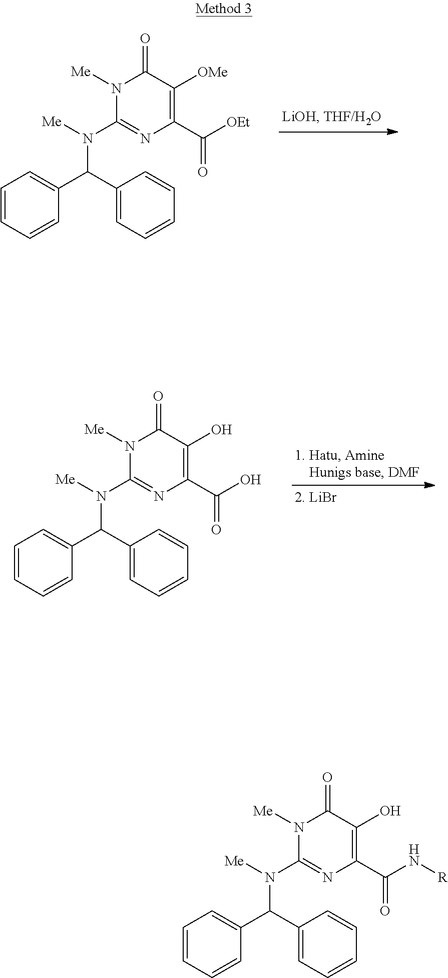

Method is a 3-step protocol for the preparation of hydroxy pyrimidine analogs starting from ethyl 2-(benzhydryl(methyl)amino)-5-methoxy-1-methyl-6-oxo-1,6-dihydropyrimidine-4-carboxylate as prepared in method 1. Method 3 applies to aryl amines including heteroaryl amines.

The representative examples that follow are intended to help illustrate the present disclosure, and are not intended to, nor should they be construed to, limit the scope of the invention.

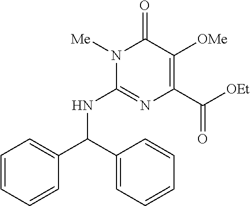 [SMILES: CCOC(=O)c1nc(NC(c2ccccc2)c2ccccc2)n(C)c(=O)c1OC]

Method 1, step 1. ethyl 2-(benzhydrylamino)-5-methoxy-1-methyl-6-oxo-1,6-dihydropyrimidine-4-carboxylate: To a 50° C. stirred solution of ethyl 2-chloro-5-methoxy-1-methyl-6-oxopyrimidine-4-carboxylate (4.0 g, 16.2 mmol) and cesium fluoride (2.4 g, 16.2 mmol) in DMF (70 mL) was added diphenylmethanamine (4.5 g, 24.3 mmol) portion wise. After stirring for 6 h the reaction mixture was diluted with EtOAc and washed with brine. The organic layer was collected and dried over Na₂SO₄ then concentrated in vacuo. The resulting crude material was purified by silica gel chromatography (2:3 EtOAc/pet. ether). Fractions containing product were combined and the solvent was removed in vacuo giving the desired product as a yellow solid (5.2 g, 70% yield). LCMS: m/z=394.1 [M+1].

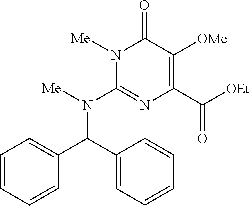 [SMILES: CCOC(=O)c1nc(N(C)C(c2ccccc2)c2ccccc2)n(C)c(=O)c1OC]

Method 1, step 2. ethyl 2-(benzhydryl(methyl)amino)-5-methoxy-1-methyl-6-oxo-1,6-dihydropyrimidine-4-carboxylate: To a stirred solution of ethyl 2-[(diphenylmethyl)amino]-5-methoxy-1-methyl-6-oxopyrimidine-4-carboxylate (4.2 g, 10.7 mmol) and cesium carbonate (7.0 g, 21.4 mmol) in DMF (100 mL) was added methyl iodide (4.6 g, 32.1 mmol) portion wise. After stirring for 4 h the reaction mixture was diluted with EtOAc and washed with brine. The organic layer was then collected, dried over Na₂SO₄ then concentrated in vacuo. The resulting crude material was purified by reverse phase C18 chromatography (4:1 Acetonitrile/water (0.1% FA). Fractions containing product were combined and the solvent was removed in vacuo giving the desired product as a yellow solid (3.1 g, 66% yield). LCMS: m/z=408.3 [M+1].

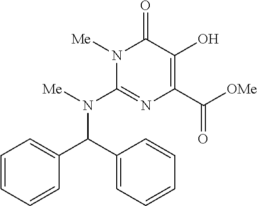 [SMILES: COC(=O)c1nc(N(C)C(c2ccccc2)c2ccccc2)n(C)c(=O)c1O]

Method 2, step 1. methyl 2-(benzhydryl(methyl)amino)-5-hydroxy-1-methyl-6-oxo-1,6-dihydropyrimidine-4-carboxylate: To a stirred solution of ethyl 2-[(diphenylmethyl)(methyl)amino]-5-methoxy-1-methyl-6-oxopyrimidine-4-carboxylate (2.0 g, 4.90 mmol) in DMF (80 mL) was added solid lithium bromide (6.4 g, 73.5 mmol). The resulting mixture was heated to 80° C. and stirred for 8 h. This reaction mixture was then diluted with EtOAc and washed with brine, dried over Na₂SO₄ and concentrated in vacuo. The crude material was purified by reverse phase C18 chromatography (1:1 Acetonitrile/water (0.1% FA). Fractions containing product were combined and the solvent was removed in vacuo giving the desired product as an off-white solid (1 g, 50% yield)¹H NMR (400 MHz, DMSO-d₆) δ 9.87 (s, 1H), 7.47-7.39 (m, 4H), 7.27 (dd, J=8.3, 6.9 Hz, 4H), 7.22-7.13 (m, 2H), 5.58 (s, 1H), 4.22 (q, J=7.1 Hz, 2H), 3.64 (s, 3H), 2.49 (s, 3H), 1.27 (t, J=7.1 Hz, 3H). LCMS: m/z=394.3 [M+1].

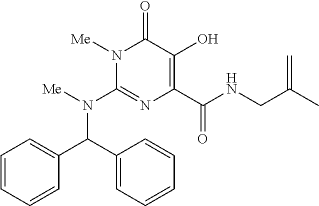 [SMILES: C=C(C)CNC(=O)c1nc(N(C)C(c2ccccc2)c2ccccc2)n(C)c(=O)c1O]

Method 2, step 2. 2-(benzhydryl(methyl)amino)-5-hydroxy-1-methyl-N-(2-methylallyl)-6-oxo-1,6-dihydropyrimidine-4-carboxamide: A microwave vial was charged with ethyl 2-[(diphenylmethyl)(methyl)amino]-5-hydroxy-1-methyl-6-oxo-1,6-dihydropyrimidine-4-carboxylate (30 mg, 76.2 μmol) and 2-methylprop-2-en-1-amine (62.1 mg, 874 μmol). This mixture was then dissolved in DMF (0.9 mL), the reaction vessel was sealed and heated to 100° C. After stirring overnight formation of the desired product was observed by LCMS. The crude reaction mixture was then purified by prep HPLC. Fractions containing product were combined and the solvent was removed in vacuo giving the desired product (18.34 mg, 57% yield). ¹H NMR (400 MHz, DMSO-d6) δ 8.59 (s, 1H), 7.52-7.44 (m, 4H), 7.25 (dd, J=8.3, 6.9 Hz, 4H), 7.19-7.10 (m, 2H), 5.80 (s, 1H), 4.87-4.79 (m, 1H), 4.70 (t, J=1.4 Hz, 1H), 3.82 (d, J=6.3 Hz, 2H), 3.64 (s, 3H), 2.51 (s, 3H), 1.70 (s, 3H).

TABLE 1

| | | | Exact |
|---|---|---|---|
| Example | | | Mass |
| Nos. | Structure | IUPAC Name | [M + 1] |
| 1 | 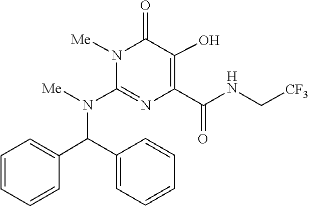 [SMILES: CN(c1nc(C(=O)NCC(F)(F)F)c(O)c(=O)n1C)C(c1ccccc1)c1ccccc1] | 2-(benzhydryl(methyl)amino)-5- hydroxy-1-methyl-6-oxo-N-(2,2,2- trifluoroethyl)-1,6- dihydropyrimidine-4-carboxamide | Calc'd 447.2, Found 447.1 |
| 2 | 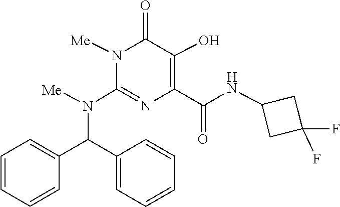 [SMILES: CN(c1nc(C(=O)NC2CC(F)(F)C2)c(O)c(=O)n1C)C(c1ccccc1)c1ccccc1] | 2-(benzhydryl(methyl)amino)-N- (3,3-difluorocyclobutyl)-5- hydroxy-1-methyl-6-oxo-1,6- dihydropyrimidine-4-carboxamide | Calc'd 455.2, Found 455.0 |
| 3 | 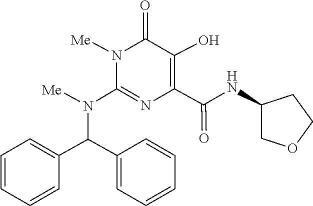 [SMILES: CN(c1nc(C(=O)N[C@H]2CCOC2)c(O)c(=O)n1C)C(c1ccccc1)c1ccccc1] | (S)-2-(benzhydryl(methyl)amino)- 5-hydroxy-1-methyl-6-oxo-N- (tetrahydrofuran-3-yl)-1,6- dihydropyrimidine-4-carboxamide | Calc'd 435.2, Found 435.1 |
| 4 | 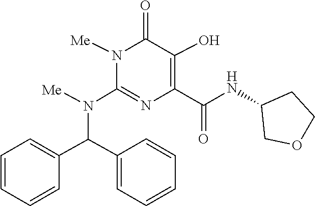 [SMILES: CN(c1nc(C(=O)N[C@@H]2CCOC2)c(O)c(=O)n1C)C(c1ccccc1)c1ccccc1] | (R)-2-(benzhydryl(methyl)amino)- 5-hydroxy-1-methyl-6-oxo-N- (tetrahydrofuran-3-yl)-1,6- dihydropyrimidine-4-carboxamide | Calc'd 435.2, Found 435.1 |
| 5 | 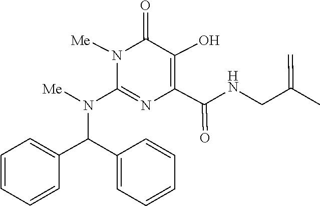 [SMILES: C=C(C)CNC(=O)c1nc(N(C)C(c2ccccc2)c2ccccc2)n(C)c(=O)c1O] | 2-(benzhydryl(methyl)amino)-5- hydroxy-1-methyl-N-(2- methylallyl)-6-oxo-1,6- dihydropyrimidine-4-carboxamide | Calc'd 419.2, Found 419.1 |
| 6 | 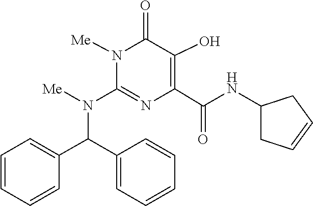 [SMILES: CN(c1nc(C(=O)NC2CC=CC2)c(O)c(=O)n1C)C(c1ccccc1)c1ccccc1] | 2-(benzhydryl(methyl)amino)-N- (cyclopent-3-en-1-yl)-5-hydroxy-1- methyl-6-oxo-1,6- dihydropyrimidine-4-carboxamide | Calc'd 431.2, Found 431.1 |
| 7 | 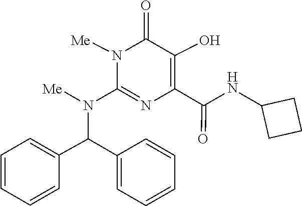 [SMILES: CN(c1nc(C(=O)NC2CCC2)c(O)c(=O)n1C)C(c1ccccc1)c1ccccc1] | 2-(benzhydryl(methyl)amino)-N- cyclobutyl-5-hydroxy-1-methyl-6- oxo-1,6- dihydropyrimidine-4-carboxamide | Calc'd 419.2, Found 419.1 |
| 8 | 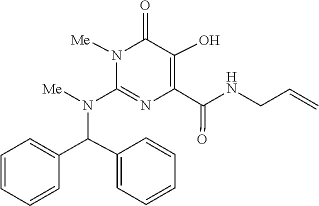 [SMILES: C=CCNC(=O)c1nc(N(C)C(c2ccccc2)c2ccccc2)n(C)c(=O)c1O] | N-allyl-2- (benzhydryl(methyl)amino)-5- hydroxy-1-methyl-6-oxo-1,6- dihydropyrimidine-4-carboxamide | Calc'd 405.2, Found 405.2 |
| 9 | 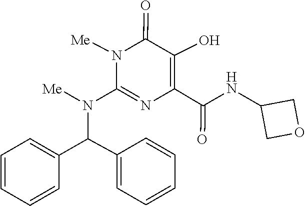 [SMILES: CN(c1nc(C(=O)NC2COC2)c(O)c(=O)n1C)C(c1ccccc1)c1ccccc1] | 2-(benzhydryl(methyl)amino)-5- hydroxy-1-methyl-N-(oxetan-3-yl)- 6-oxo-1,6- dihydropyrimidine-4-carboxamide | Calc'd 421.2, Found 421.2 |
| 10 | 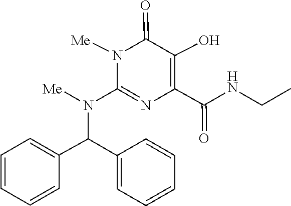 [SMILES: CCNC(=O)c1nc(N(C)C(c2ccccc2)c2ccccc2)n(C)c(=O)c1O] | 2-(benzhydryl(methyl)amino)-N- ethyl-5-hydroxy-1-methyl-6-oxo- 1,6- dihydropyrimidine-4-carboxamide | Calc'd 393.2, Found 393.9 |
| 11 | 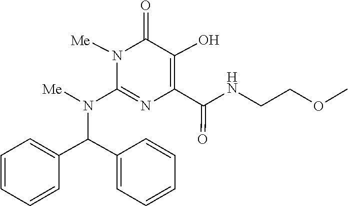 [SMILES: COCCNC(=O)c1nc(N(C)C(c2ccccc2)c2ccccc2)n(C)c(=O)c1O] | 2-(benzhydryl(methyl)amino)-5- hydroxy-N-(2-methoxyethyl)-1- methyl-6-oxo-1,6- dihydropyrimidine-4-carboxamide | Calc'd 423.2, Found 423.9 |
| 12 | 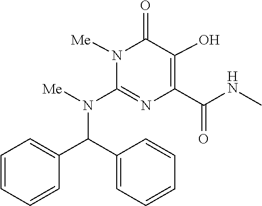 [SMILES: CNC(=O)c1nc(N(C)C(c2ccccc2)c2ccccc2)n(C)c(=O)c1O] | 2-(benzhydryl(methyl)amino)-5- hydroxy-N,1-dimethyl-6-oxo-1,6- dihydropyrimidine-4-carboxamide | Calc'd 379.2, Found 379.9 |
| 13 | 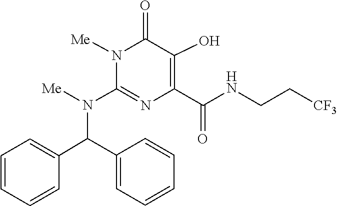 [SMILES: CN(c1nc(C(=O)NCCC(F)(F)F)c(O)c(=O)n1C)C(c1ccccc1)c1ccccc1] | 2-(benzhydryl(methyl)amino)-5- hydroxy-1-methyl-6-oxo-N-(3,3,3- trifluoropropyl)-1,6- dihydropyrimidine-4-carboxamide | Calc'd 461.2, Found 461.0 |
| 14 | 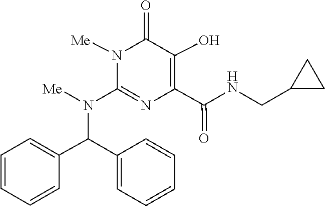 [SMILES: CN(c1nc(C(=O)NCC2CC2)c(O)c(=O)n1C)C(c1ccccc1)c1ccccc1] | 2-(benzhydryl(methyl)amino)-N- (cyclopropylmethyl)-5-hydroxy-1- methyl-6-oxo-1,6- dihydropyrimidine-4-carboxamide | Calc'd 419.2, Found 419.1 |

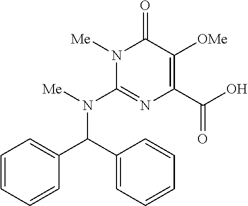 [SMILES: COc1c(C(=O)O)nc(N(C)C(c2ccccc2)c2ccccc2)n(C)c1=O]

Method 3, step 1. 2-(benzhydryl(methyl)amino)-5-methoxy-1-methyl-6-oxo-1,6-dihydropyrimidine-4-carboxylic acid: To a stirred solution of ethyl 2-[(diphenylmethyl)(methyl)amino]-5-methoxy-1-methyl-6-oxopyrimidine-4-carboxylate (1.5 g, 3.60 mmol) in THF (10 mL) was added lithium hydroxide (264.48 mg, 11.0 mmol) and water (5.00 mL). This resulting mixture was stirred for 6 h at room temperature. Upon completion the aqueous layer was washed with EtOAc and then brought to pH 6 using HCl (aq). The product was then extracted with CH₂Cl₂. Concentration of the organic layer in vacuo resulted in the desired product as an off-white solid (1.4 g, 98% yield).

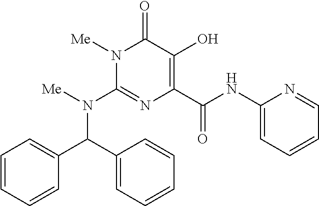 [SMILES: CN(c1nc(C(=O)Nc2ccccn2)c(O)c(=O)n1C)C(c1ccccc1)c1ccccc1]

Method 3, step 1. 2-(benzhydryl(methyl)amino)-5-hydroxy-1-methyl-6-oxo-N-(pyridin-2-yl)-1,6-dihydropyrimidine-4-carboxamide: To a stirred solution of 2-[(diphenylmethyl)(methyl)amino]-5-methoxy-1-methyl-6-oxo-1,6-dihydropyrimidine-4-carboxylic acid (18.9 mg, 0.05 mmol) in DMF (0.5 mL) was added Hatu (28.5 mg, 0.075 mmol) and Hunigs base (0.026 mL, 0.150 mmol) followed by pyridin-2-amine (4.7 mg, 0.05 mmol). This reaction was stirred at rt for 1 hour at which point complete conversion to the desired amide was observed by LCMS. Lithium bromide (43.4 mg, 0.5 mmol) and water (0.10 mL) was added to the reaction and the mixture was heated to 70° C. and monitored by LCMS. After stirring at temp for 4 hours additional lithium bromide (22 mg, 0.25 mmol) was added and the reaction continued to stir at 70° C. overnight. Upon complete conversion to the desired product as observed by LCMS the reaction was cooled to rt and diluted with CH₂Cl₂, washed with brine then concentrated in vacuo. The crude reaction material was purified by prep HPLC, fractions containing product were combined then concentrated in vacuo giving the desired product as a white solid (6.3 mg, 28% yield). ¹H NMR (400 MHz, DMSO-d6) δ=8.47 (d, J=4.9 Hz, 1H), 8.14-8.03 (m, 1H), 7.96-7.74 (m, 2H), 7.51 (d, J=7.8 Hz, 5H), 7.33-7.19 (m, 7H), 7.19-7.04 (m, 3H), 5.69 (s, 1H), 3.32-3.22 (m, 4H), 2.56 (s, 3H)

TABLE 2

| | | | Exact |
|---|---|---|---|
| Example | | | Mass |
| Nos. | Structure | IUPAC Name | [M + 1] |
| 15 | 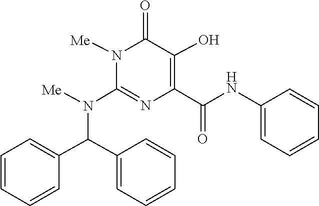 [SMILES: CN(c1nc(C(=O)Nc2ccccc2)c(O)c(=O)n1C)C(c1ccccc1)c1ccccc1] | 2-(benzhydryl(methyl)amino)-5- hydroxy-1-methyl-6-oxo-N- phenyl-1,6- dihydropyrimidine-4-carboxamide | Calc'd 441.2, Found 441.1 |
| 16 | 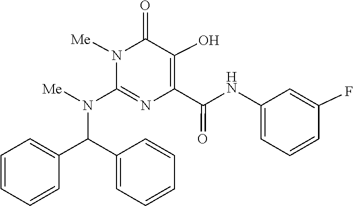 [SMILES: CN(c1nc(C(=O)Nc2cccc(F)c2)c(O)c(=O)n1C)C(c1ccccc1)c1ccccc1] | 2-(benzhydryl(methyl)amino)-N- (3-fluorophenyl)-5-hydroxy-1- methyl-6-oxo-1,6- dihydropyrimidine-4-carboxamide | Calc'd 459.2, Found 459.1 |
| 17 | 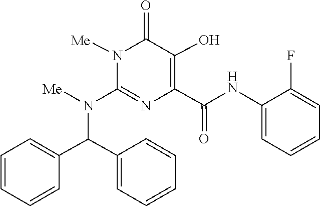 [SMILES: CN(c1nc(C(=O)Nc2ccccc2F)c(O)c(=O)n1C)C(c1ccccc1)c1ccccc1] | 2-(benzhydryl(methyl)amino)-N- (2-fluorophenyl)-5-hydroxy-1- methyl-6-oxo-1,6- dihydropyrimidine-4-carboxamide | Calc'd 459.2, Found 459.2 |
| 18 | 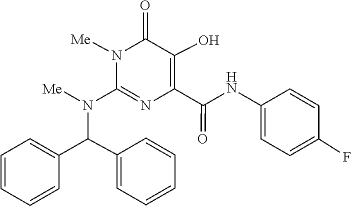 [SMILES: CN(c1nc(C(=O)Nc2ccc(F)cc2)c(O)c(=O)n1C)C(c1ccccc1)c1ccccc1] | 2-(benzhydryl(methyl)amino)-N- (4-fluorophenyl)-5-hydroxy-1- methyl-6-oxo-1,6- dihydropyrimidine-4-carboxamide | Calc'd 459.2, Found 459.6 |
| 19 | 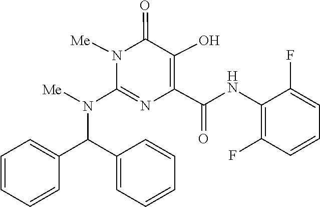 [SMILES: CN(c1nc(C(=O)Nc2c(F)cccc2F)c(O)c(=O)n1C)C(c1ccccc1)c1ccccc1] | 2-(benzhydryl(methyl)amino)-N- (2,6-difluorophenyl)-5-hydroxy-1- methyl-6-oxo-1,6- dihydropyrimidine-4-carboxamide | Calc'd 477.2, Found 477.6 |
| 20 | 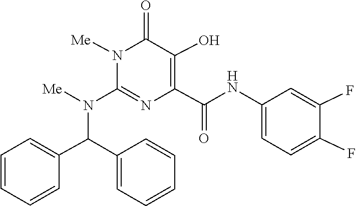 [SMILES: CN(c1nc(C(=O)Nc2ccc(F)c(F)c2)c(O)c(=O)n1C)C(c1ccccc1)c1ccccc1] | 2-(benzhydryl(methyl)amino)-N- (3,4-difluorophenyl)-5-hydroxy-1- methyl-6-oxo-1,6- dihydropyrimidine-4-carboxamide | Calc'd 477.2, Found 477.7 |
| 21 | 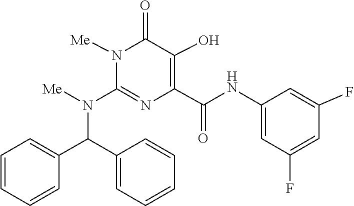 [SMILES: CN(c1nc(C(=O)Nc2cc(F)cc(F)c2)c(O)c(=O)n1C)C(c1ccccc1)c1ccccc1] | 2-(benzhydryl(methyl)amino)-N- (3,5-difluorophenyl)-5-hydroxy-1- methyl-6-oxo-1,6- dihydropyrimidine-4-carboxamide | Calc'd 477.2, Found 477.2 |
| 22 | 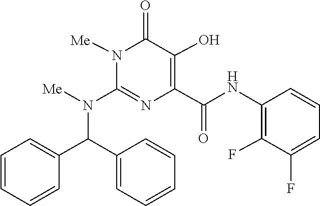 [SMILES: CN(c1nc(C(=O)Nc2cccc(F)c2F)c(O)c(=O)n1C)C(c1ccccc1)c1ccccc1] | 2-(benzhydryl(methyl)amino)-N- (2,3-difluorophenyl)-5-hydroxy-1- methyl-6-oxo-1,6- dihydropyrimidine-4-carboxamide | Calc'd 477.2, Found 477.2 |
| 23 | 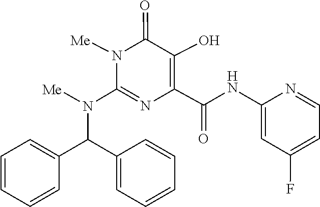 [SMILES: CN(c1nc(C(=O)Nc2cc(F)ccn2)c(O)c(=O)n1C)C(c1ccccc1)c1ccccc1] | 2-(benzhydryl(methyl)amino)-N- (4-fluoropyridin-2-yl)-5-hydroxy- 1-methyl-6-oxo-1,6- dihydropyrimidine-4-carboxamide | Calc'd 460.2 Found 460.2 |
| 24 | 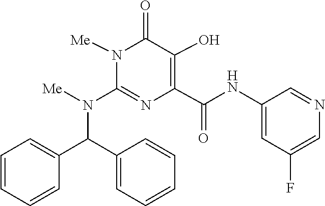 [SMILES: CN(c1nc(C(=O)Nc2cncc(F)c2)c(O)c(=O)n1C)C(c1ccccc1)c1ccccc1] | 2-(benzhydryl(methyl)amino)-N- (5-fluoropyridin-3-yl)-5-hydroxy- 1-methyl-6-oxo-1,6- dihydropyrimidine-4-carboxamide | Calc'd 460.2, Found 460.2 |
| 25 | 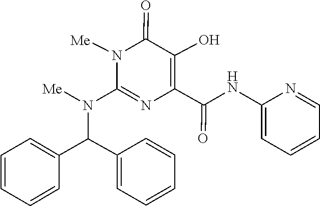 [SMILES: CN(c1nc(C(=O)Nc2ccccn2)c(O)c(=O)n1C)C(c1ccccc1)c1ccccc1] | 2-(benzhydryl(methyl)amino)-5- hydroxy-1-methyl-6-oxo-N- (pyridin-2-yl)-1,6- dihydropyrimidine-4-carboxamide | Calc'd 442.2, Found 442.2 |
| 26 | 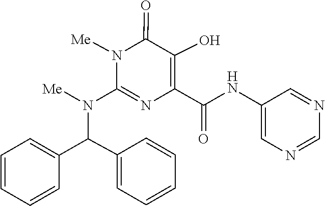 [SMILES: CN(c1nc(C(=O)Nc2cncnc2)c(O)c(=O)n1C)C(c1ccccc1)c1ccccc1] | 2-(benzhydryl(methyl)amino)-5- hydroxy-1-methyl-6-oxo-N- (pyrimidin-5-yl)-1,6- dihydropyrimidine-4-carboxamide | Calc'd 443.2, Found 443.1 |
| 27 | 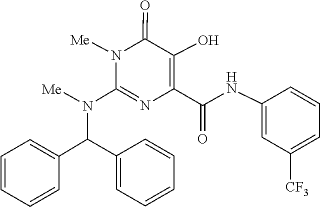 [SMILES: CN(c1nc(C(=O)Nc2cccc(C(F)(F)F)c2)c(O)c(=O)n1C)C(c1ccccc1)c1ccccc1] | 2-(benzhydryl(methyl)amino)-5- hydroxy-1-methyl-6-oxo-N-(3- (trifluoromethyl)phenyl)-1,6- dihydropyrimidine-4-carboxamide | Calc'd 509.2, Found 509.2 |
| 28 | 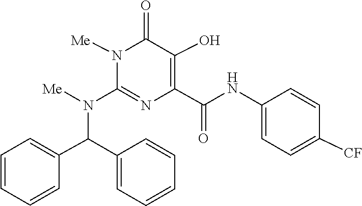 | 2-(benzhydryl(methyl)amino)-5- hydroxy-1-methyl-6-oxo-N-(4- (trifluoromethyl)phenyl)-1,6- dihydropyrimidine-4-carboxamide | Calc'd 509.2, Found 509.1 |
| 29 | 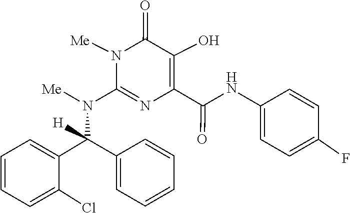 | 2-(((2- chlorophenyl)(phenyl)methyl) (methyl)amino)-N-(4-fluorophenyl)- 5-hydroxy-1-methyl-6-oxo-1,6- dihydropyrimidine-4-carboxamide | Calc'd 493.1, Found 493.7 |
| 30 | 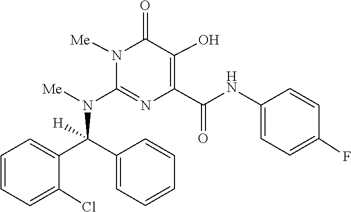 [SMILES: CN(c1nc(C(=O)Nc2ccc(F)cc2)c(O)c(=O)n1C)[C@@H](c1ccccc1)c1ccccc1Cl] | (S)-2-(((2- chlorophenyl)(phenyl)methyl) (methyl)amino)-N-(4-fluorophenyl)- 5-hydroxy-1-methyl-6-oxo-1,6- dihydropyrimidine-4-carboxamide | Calc'd 493.1, Found 493.7 |
| 31 | 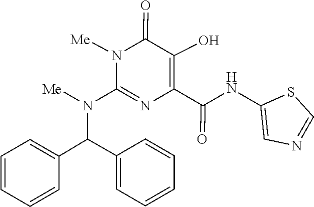 [SMILES: CN(c1nc(C(=O)Nc2cncs2)c(O)c(=O)n1C)C(c1ccccc1)c1ccccc1] | 2-(benzhydryl(methyl)amino)-5- hydroxy-1-methyl-6-oxo-N- (thiazol-5-yl)-1,6- dihydropyrimidine-4-carboxamide | Calc'd 447.1, Found 447.3 |
| 32 | 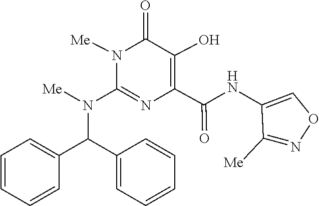 [SMILES: Cc1nocc1NC(=O)c1nc(N(C)C(c2ccccc2)c2ccccc2)n(C)c(=O)c1O] | 2-(benzhydryl(methyl)amino)-5- hydroxy-1-methyl-N-(3- methylisoxazol-4-yl)-6-oxo-1,6- dihydropyrimidine-4-carboxamide | Calc'd 445.2 Found 445.3 |
| 33 | 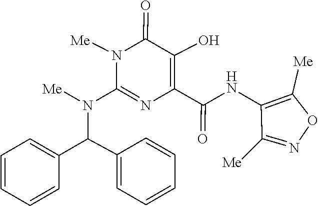 [SMILES: Cc1noc(C)c1NC(=O)c1nc(N(C)C(c2ccccc2)c2ccccc2)n(C)c(=O)c1O] | 2-(benzhydryl(methyl)amino)-N- (3,5-dimethylisoxazol-4-yl)-5- hydroxy-1-methyl-6-oxo-1,6- dihydropyrimidine-4-carboxamide | Calc'd 459.2 Found 459.4 |
| 34 | 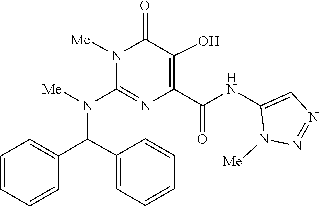 [SMILES: CN(c1nc(C(=O)Nc2cnnn2C)c(O)c(=O)n1C)C(c1ccccc1)c1ccccc1] | 2-(benzhydryl(methyl)amino)-5- hydroxy-1-methyl-N-(1-methyl- 1H-1,2,3-triazol-5-yl)-6-oxo-1,6- dihydropyrimidine-4-carboxamide | Calc'd 445.2 Found 445.3 |
| 35 | 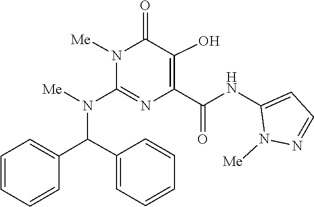 [SMILES: CN(c1nc(C(=O)Nc2ccnn2C)c(O)c(=O)n1C)C(c1ccccc1)c1ccccc1] | 2-(benzhydryl(methyl)amino)-5- hydroxy-1-methyl-N-(1-methyl- 1H-pyrazol-5-yl)-6-oxo-1,6- dihydropyrimidine-4-carboxamide | Calc'd 444.2 Found 444.5 |
| 36 | 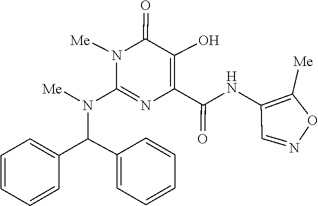 [SMILES: Cc1oncc1NC(=O)c1nc(N(C)C(c2ccccc2)c2ccccc2)n(C)c(=O)c1O] | 2-(benzhydryl(methyl)amino)-5- hydroxy-1-methyl-N-(5- methylisoxazol-4-yl)-6-oxo-1,6- dihydropyrimidine-4-carboxamide | Calc'd 445.2 Found |
| 37 | 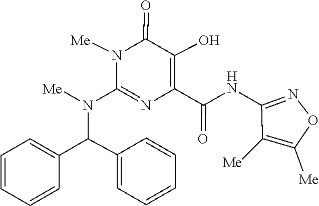 [SMILES: Cc1onc(NC(=O)c2nc(N(C)C(c3ccccc3)c3ccccc3)n(C)c(=O)c2O)c1C] | 2-(benzhydryl(methyl)amino)-N- (4,5-dimethylisoxazol-3-yl)-5- hydroxy-1-methyl-6-oxo-1,6- dihydropyrimidine-4-carboxamide | Calc'd 459.2 Found |
| 38 | 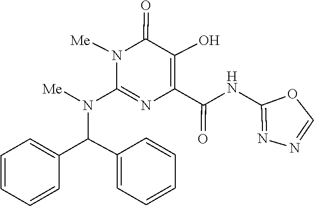 [SMILES: CN(c1nc(C(=O)Nc2nnco2)c(O)c(=O)n1C)C(c1ccccc1)c1ccccc1] | 2-(benzhydryl(methyl)amino)-5- hydroxy-1-methyl-N-(1,3,4- oxadiazol-2-yl)-6-oxo-1,6- dihydropyrimidine-4-carboxamide | Calc'd 432.2 Found |
| 39 | 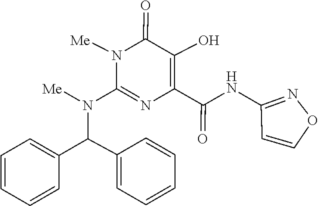 [SMILES: CN(c1nc(C(=O)Nc2ccon2)c(O)c(=O)n1C)C(c1ccccc1)c1ccccc1] | 2-(benzhydryl(methyl)amino)-5- hydroxy-N-(isoxazol-3-yl)-1- methyl-6-oxo-1,6- dihydropyrimidine-4-carboxamide | Calc'd 431.2 Found |

### Biochemical Assays

### 1. Silencing TREX1 in Tumor Cells

Activation of the cGAS/STING pathway upon sensing of cytosolic DNA and subsequent type I IFN production can occur in both tumor cells and innate immune cells, particularly dendritic cells. To evaluate whether TREX1 keeps in check the production of type I IFN by a well described, cold syngeneic tumor model that undergoes immune-mediated rejection upon activation of type I IFN by STING agonists, TREX1 was knocked down in B16F10 tumor cells using CRISPR (FIG. 1A). Accumulation of cytosolic DNA via DNA transfection of the tumor cells resulted in an about 5-fold increase in IFNβ production by the TREX1 knockout B16F10 cells relative to the parental tumor cells, demonstrating that TREX1 attenuated the activation of the cGAS/STING pathway in B16F10 tumor cells (FIG. 1B).

### 2. Growth of TREX1-Competent and -Deficient B16F10 Tumor Cells In Vivo

The growth of TREX1-competent and -deficient B16F10 tumor cells in vivo was evaluated. C₅₇BL/6J mice were inoculated subcutaneously on the right flank with 300,000 parental or TREX1 knockout B16F10 tumor cells. Body weights were collected two times per week, and tumor measurements, two to three times per week, starting when tumors became measurable and for the remaining duration of the study. Tumors in which TREX1 had been silenced presented with remarkably smaller volumes than the parental B16F10 tumors (FIG. 2).

Tumors were harvested on day 19, upon termination of the study, and digested into single cell suspensions to enable flow cytometry quantification of tumor-infiltrating immune populations. TREX1 knockout B16F10 tumors were found to exhibit a significant increase in overall immune cells, which reflected an increase in the number of tumor infiltrating CD4 and CD8 T cells as well as in plasmacytoid dendritic cells (pDCs) (FIG. 3). pDCs are known to play a central role in the induction of antigen-specific anti-tumor immune responses whereas T cells are known to be major effectors of anti-tumor efficacy in mice and humans. The profound change in the immune infiltrate of the tumors deficient in TREX1 thus suggest that the inhibition of the growth of the latter tumors is at least in part immune-mediated.

### TREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length human TREX1 (expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 150 pM TREX1, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction were 112.5 pM TREX1, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

### TREX2 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged human TREX2 (residues M44-A279, expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (IDT)) in reaction buffer. Final concentrations were 2.5 nM TREX2, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction mixture were 1.875 pM TREX2, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

Results are shown in Table 1. TREX1 IC₅₀: A=0.1 μM; B=0.1 to 1 μM; C=1 to 10 μM; D=>10 μM. TREX2 IC₅₀: A=<1 μM, B=1 to 10 μM, C=10 to 100 μM, D=>100 μM.

TABLE 3

| Example | TREX1 IC₅₀ | TREX2 IC₅₀ |
|---|---|---|
| 1 | C | C |
| 2 | C | C |
| 3 | D | C |
| 4 | D | C |
| 5 | C | C |
| 6 | C | C |
| 7 | C | C |
| 8 | C | B |
| 9 | D | C |
| 10 | C | C |
| 11 | D | C |
| 12 | D | C |
| 13 | B | C |
| 14 | B | B |
| 15 | A | A |
| 16 | A | A |
| 17 | A | A |
| 18 | B | A |
| 19 | C | C |
| 20 | B | A |
| 21 | B | A |
| 22 | A | A |
| 23 | C | B |
| 24 | B | B |
| 25 | B | A |
| 26 | C | B |
| 27 | D | C |
| 28 | D | C |
| 29 | B | A |
| 30 | B | A |
| 31 | B | B |
| 32 | A | B |
| 33 | B | C |
| 34 | C | B |
| 35 | C | C |
| 36 | B | B |
| 37 | C | C |
| 38 | C | C |
| 39 | B | C |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 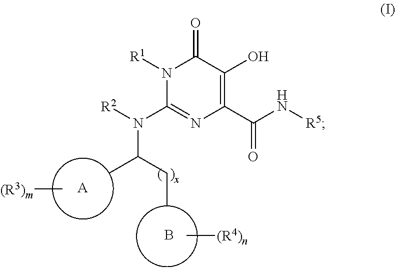
or a pharmaceutically acceptable salt thereof, wherein:
R¹ is hydrogen, (C₁-C₄)alkyl, halo(C₁-C₄)alkyl, or 3- to 4-membered cycloalkyl;
R² is hydrogen or (C₁-C₄)alkyl optionally substituted with phenyl, wherein said phenyl is optionally substituted with 1 to 3 groups selected from halo, (C₁-C₄)alkyl, and halo(C₁-C₄)alkyl;
Ring A and Ring B are each independently is aryl, heteroaryl, heterocyclyl, or cycloalkyl;
R³, R⁴, and R⁶ are each independently (C₁-C₆)alkyl, halo(C₁-C₆)alkyl, (C₁-C₆)alkylOR^{b}, (C₂-C₆)alkenyl, halo(C₁-C₆)alkoxy, halo, phenyl, —CN, —NR^{a}C(O)OR^{b}, —NR^{a}C(S)OR^{b}, —C(O)R^{b}, —NR^{a}C(O)NR^{b}R^{g}, —NR^{a}C(S)NR^{b}R^{g}, —NR^{a}S(O)₂NR^{b}R^{g}, —C(S)R^{b}, —S(O)₂R^{c}, —S(O)R^{c}, —C(O)OR^{d}, —C(S)OR^{d}, —C(O)NR^{e}R^{f}, —C(S)NR^{a}R^{e}, —NR^{a}C(O)R^{d}, —NR^{a}C(S)R^{d}, —OR^{e}, —SR^{e}, —O(C₁-C₄)alkylOR^{e}, —NR^{e}R^{f}, 4- to 6-membered heteroaryl, or 4- to 7-membered heterocyclyl, wherein
i) said phenyl for R³ and R⁴ are each independently optionally substituted with 1 or 2 groups selected from R^{g};
ii) said (C₁-C₆)alkyl for R³ and R⁴ are each independently optionally substituted with 1 or 2 groups selected from OR^{h}, —NR^{j}R^{k}, phenyl, and 5- to 6-membered heteroaryl; and
ii) said 4- to 7-membered heterocyclyl and 4- to 6-membered heteroaryl for R³ and R⁴ are each independently optionally substituted with 1 or 2 groups selected from R^{m}; and
iv) said phenyl and 5- to 6-membered heteroaryl of the optional substituents listed for (C₁-C₆)alkyl in R³ and R⁴ are each independently optionally substituted with 1 or 2 groups selected from R^{g};
x is 0, 1, or 2;
m and n are each independently an integer from 0 to 3;
R⁵ is (C₁-C₆)alkyl, halo(C₁-C₆)alkyl, (C₂-C₆)alkenyl, —(C₁-C₆)alkylOR^{a}, —(C₁-C₆)alkylNR^{a}R^{b}, —(C₁-C₆)alkyl(C₃-C₇)cycloalkyl, —(C₁-C₆)alkyl(4- to 7-membered heterocyclyl), —(C₁-C₆)alkyl(5- to 7-membered heteroaryl), phenyl, 5- to 7-membered heteroaryl, (C₃-C₇)cycloalkyl, (C₃-C₇)cycloalkenyl, or 4- to 7-membered heterocyclyl, wherein each occurrence of said phenyl, 5- to 7-membered heteroaryl, (C₃-C₇)cycloalkyl, (C₃-C₇)cycloalkenyl, or 4- to 7-membered heterocyclyl are optionally and independently substituted with 1 to 3 groups selected from R⁶, provided R⁵ is not an optionally substituted isoxazolyl;
R^{a}, R^{b}, R^{c}, R^{d}, R^{e} and R^{f} are each independently hydrogen, halo, (C₁-C₆)alkyl, halo(C₁-C₆)alkyl, (C₁-C₆)alkoxy, halo(C₁-C₆)alkoxy, phenyl, 3- to 4-membered cycloalkyl, 4- to 6-membered heteroaryl, or 4- to 7-membered heterocyclyl, wherein
i) said (C₁-C₆)alkyl for R^{a}, R^{b}, R^{c}, R^{d}, R^{e} and R^{f} is optionally substituted with 1 or 2 groups selected from phenyl, —OR^{h}, and —NR^{j}R^{k};
ii) said phenyl, 4- to 6-membered heteroaryl, and 4- to 7-membered heterocyclyl for R^{a}, R^{b}, R^{c}, R^{d}, R^{e}, and R^{f} are each optionally and independently substituted with 1 or 2 groups selected from R^{g};
iii) said 4- to 7-membered heterocyclyl for R^{a}, R^{b}, R^{c}, R^{d}, R^{e}, and R^{f} is further optionally substituted with ═O; and
R^{g}, R^{h}, R^{j}, R^{k}, and R^{m} are each independently hydrogen, halo, (C₁-C₆)alkyl, halo(C₁-C₆)alkyl, (C₁-C₆)alkoxy, halo(C₁-C₆)alkoxy, phenyl, —(C₁-C₆)alkylphenyl, 3- to 4-membered cycloalkyl, 4- to 6-membered heteroaryl, or 4- to 7-membered heterocyclyl, and wherein said 4- to 7-membered heterocyclyl for R^{g}, R^{h}, R^{j} and R^{k} is further optionally substituted with ═O.

2. The compound of claim 1, wherein the compound is of the Formula II: 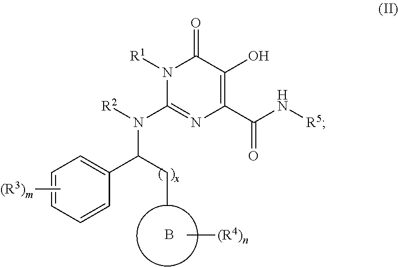
or a pharmaceutically acceptable salt thereof.

3. (canceled)

4. The compound of claim 1, wherein the compound is of the Formula IV: 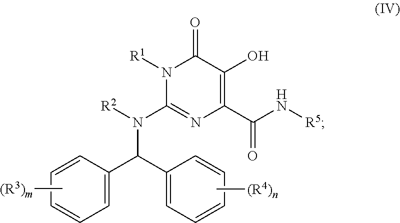
or a pharmaceutically acceptable salt thereof.

5. (canceled)

6. The compound of claim 1, wherein the compound is of the Formula V: 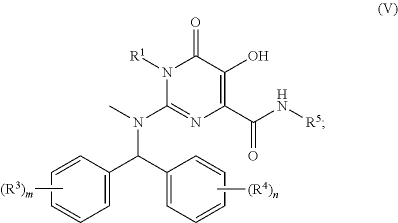
or a pharmaceutically acceptable salt thereof.

7. The compound of claim 1, or a pharmaceutically acceptable salt thereof, wherein R¹ is (C₁-C₄)alkyl.

8. The compound of claim 1, wherein the compound is of the Formula VI: 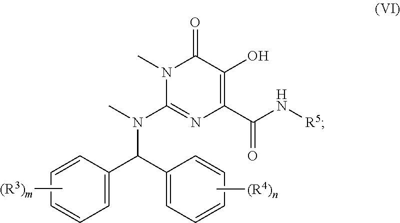
or a pharmaceutically acceptable salt thereof.

9. The compound of claim 1, or a pharmaceutically acceptable salt thereof, wherein each R³ and R⁴ are independently selected from (C₁-C₄)alkyl, halo(C₁-C₄)alkyl, (C₁-C₄)alkoxy, halo(C₁-C₄)alkoxy, halo and CN.

10. The compound of claim 1, or a pharmaceutically acceptable salt thereof, wherein R³ is halo.

11. The compound of claim 1, or a pharmaceutically acceptable salt thereof, wherein R³ is chloro.

12. The compound of claim 1, or a pharmaceutically acceptable salt thereof, wherein m is 0 or 1.

13. The compound of claim 1, or a pharmaceutically acceptable salt thereof, wherein n is 0.

14. The compound of claim 1, or a pharmaceutically acceptable salt thereof, wherein R⁵ is (C₁-C₆)alkyl, halo(C₁-C₆)alkyl, (C₂-C₆)alkenyl, —(C₁-C₆)alkylOR^{a}, —(C₁-C₆)alkyl(C₃-C₇)cycloalkyl, phenyl, 5- to 7-membered heteroaryl, (C₃-C₇)cycloalkyl, (C₃-C₇)cycloalkenyl, 4- to 7-membered heterocyclyl wherein each occurrence of said phenyl, 5- to 7-membered heteroaryl, (C₃-C₇)cycloalkyl, (C₃-C₇)cycloalkenyl, or 4- to 7-membered heterocyclyl are optionally and independently substituted with 1 to 3 groups selected from R⁶.

15. The compound of claim 1, or a pharmaceutically acceptable salt thereof, wherein R⁵ is (C₁-C₄)alkyl, halo(C₁-C₄)alkyl, (C₃-C₅)alkenyl, —(C₁-C₄)alkylO(C₁-C₄)alkyl, —(C₁-C₄)alkyl(C₄-C₆)cycloalkyl, phenyl, 5- to 6-membered heteroaryl, (C₃-C₆)cycloalkyl, (C₄-C₆)cycloalkenyl, or 4- to 6-membered heterocyclyl wherein each occurrence of said phenyl, 5- to 6-membered heteroaryl, (C₃-C₆)cycloalkyl, (C₄-C₆)cycloalkenyl, or 4- to 6-membered heterocyclyl are optionally and independently substituted with 1 to 3 groups selected from R⁶.

16. The compound of claim 1, or a pharmaceutically acceptable salt thereof, wherein R⁵ is (C₁-C₄)alkyl, halo(C₁-C₄)alkyl, (C₃-C₅)alkenyl, —(C₁-C₄)alkylO(C₁-C₄)alkyl, —(C₁-C₄)alkylcyclopropyl, cyclobutyl, cyclopentenyl, cyclobutyl, phenyl, pyridyl, pyrimidyl, oxazolyl, thiazolyl, isothiazolyl, oxadiazolyl, pyrrolyl, pyrazole or tetrazolyl, wherein each of said cyclopropyl, cyclobutyl, cyclopentenyl, cyclobutyl, phenyl, pyridyl, pyrimidyl, oxazolyl, thiazolyl, isothiazolyl, oxadiazolyl, pyrrolyl, pyrazole and tetrazolyl are optionally and independently substituted with 1 to 3 groups selected from R⁶.

17. The compound of claim 1, or a pharmaceutically acceptable salt thereof, wherein R⁵ is phenyl, pyridyl, pyrimidyl, oxazolyl, thiazolyl, isothiazolyl, oxadiazolyl, pyrrolyl, pyrazole or tetrazolyl, each of which is optionally and independently substituted with 1 to 3 groups selected from R⁶.

18. The compound of claim 1, or a pharmaceutically acceptable salt thereof, wherein R⁶ is (C₁-C₆)alkyl, halo(C₁-C₆)alkyl, (C₁-C₆)alkylOR^{b}, (C₂-C₆)alkenyl, O(C₁-C₄)alkylOR^{e}, halo(C₁-C₆)alkoxy, halo, —NR^{a}C(O)OR^{b}, —NR^{a}C(S)OR^{b}, —C(O)R^{b}, —NR^{a}C(O)NR^{b}R^{g}, —NR^{a}C(S)NR^{b}R^{g}, —NR^{a}S(O)₂NR^{b}R^{g}, —C(S)R^{b}, S(O)₂R^{b}, S(O)Rb^{c}, —C(O)OR^{b}, —C(S)OR^{b}, —C(O)NR^{e}R^{f}, —C(S)NR^{a}R^{e}, —NR^{a}C(O)R^{d}, —NR^{a}C(S)R^{b}, —OR^{e}, —SR^{e}, or —NR^{e}R^{f}.

19. The compound of claim 1, or a pharmaceutically acceptable salt thereof, wherein R^{a}, R^{b}, R^{c}, R^{e}, R^{f}, and R^{g} are each independently hydrogen, (C₁-C₆)alkyl or halo(C₁-C₆)alkyl.

20. The compound of claim 1, or a pharmaceutically acceptable salt thereof, wherein R⁶ is (C₁-C₄)alkyl or halo(C₁-C₄)alkyl.

21. A pharmaceutical composition comprising the compound of claim 1, or a pharmaceutically acceptable salt thereof; and a pharmaceutically acceptable carrier.

22. A method of treating a disease responsive to the inhibition of TREX1 in a subject, comprising administering to the subject, a therapeutically effective amount of a compound of claim 1, or a pharmaceutically acceptable salt thereof.

23. (canceled)
